(19) United States
(12) Patent Application Publication
(10) Pub. No.: US 2009/0176258 A1
(43) Pub. Date: Jul. 9, 2009

(54) TEST FOR THE DETECTION OF PATHOLOGICAL PRIONS

(76) Inventor: Reinhard Latza, St. Ingbert (DE)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 12/281,892

(22) PCT Filed: Mar. 3, 2007
(86) PCT No.: PCT/EP2007/001844

§ 371 Date: Dec. 15, 2008

(30) Foreign Application Priority Data
Mar. 6, 2006 (DE) 10 2006 010 647.4

(51) Int. Cl. G01N 33/573; G01N 33/53

(52) U.S. Cl. 435/7.92; 435/7.1

(57) ABSTRACT

The am of the invention is to create a method for detecting pathological prions which is highly sensitive, can be carried out quickly and at a low cost, and allows prions to be detected at an early stage of a disease. Said aim is achieved by the fact that immobilized capture antibodies bind the pathological and non-pathological form of the prion protein contained in a sample whereupon the bound non-pathological form is specifically divided by means of plasmin. The undivided, pathological form of the prion protein, which is bound by the immobilized capture antibodies, can then be easily detected with the aid of detection antibodies. The inventive method is used for detecting pathological prions.

The present invention relates to a method for detecting pathological prions in vitro in a sample and a diagnostic kit for performing this method.

Transmissible spongiform encephalopathies (TSE) or prion diseases are degenerative brain diseases, which are accompanied by characteristic spongy histological changes in the brain and always have a fatal outcome. According to the Prusiner theory (Science 1982, 216:136-144), the agent of these diseases is an infectious protein without a detectable nucleic acid, the prion (“proteinaceous infectious agent”). It concerns a misfolded form (PrP^{Sc}, Sc: “Scrapie”) of a naturally occurring protein, the cellular prion protein (PrP^{C}). The multiplication of the causal agent takes place through transformation of the normal structure of the prion protein into the misfolded form, the occurrence of which is associated with the infection or the disease. In agreement with this hypothesis, mouse strains lacking the prion protein cannot be infected experimentally. Consequently, the TSE causal agents are often also referred to as prions and the overall category of these syndromes is grouped together as prion diseases.

Spongiform encephalopathies occur with many mammals including man. In the case of man, it is Creutzfeldt-Jakob Disease (CJD), Gerstmann-Sträussler-Scheinker Syndrome (GSS), Fatal Familial Insomnia (FFI), Kuru and the variant of Creutzfeldt-Jakob disease (vCJD). Scrapie in sheep has been known the longest. Since 1984, bovine spongiform encephalopathy (BSE) and, since 1996, the variant of Creutzfeldt-Jakob Disease have been documented. Over 180,000 head of cattle have since contracted the disease bovine spongiform encephalopathy (BSE) and been slaughtered in Great Britain and other EU countries. BSE has been detected in over 290 head of cattle in Germany.

For the first time, in 1993, two young British farmers contracted an unusual form of Creutzfeldt-Jakob Disease (CJD), which was described in 1996 as the new variant of CJD (vCJD). Up to the present day, over a hundred people in Great Britain have fallen to this disease. Today, it can safely be assumed that it involves BSE in man.

In view of the fatal outcome, the transmissibility to man, the long incubation times and the absence of therapies, the diagnosis of transmissible spongiform encephalopathies is of the greatest importance.

Three BSE quick tests have so far received the EU permit (EUROPEAN COMMISSION (1999) DIRECTORATE-GENERAL XXIV CONSUMER POLICY AND CONSUMER HEALTH PROTECTION Directorate B—Scientific Health Opinions The Evaluation of Tests for the Diagnosis of transmissible spongiform encephalopathy in bovines www.eu-komission.de):
Prionics Check, originally developed by the firm Prionics AG (Zurich, Switzerland), has been marketed worldwide by Roche Diagnostics since 1 Feb. 2001. The test is based on the Western blot, the test duration being seven to eight hours.
Platelia® BSE test is marketed by the firm Bio-Rad Laboratories (USA) and was developed jointly with the Commission de L'Energie Atomique (France). It is based on an ELISA; the test duration is four to seven hours.
Enfer TSE from the firm Enfer Technology (Ireland) is based on the ELISA principle; the test duration is four hours.

All three tests employ prion-specific antibodies against the fragment PrP²⁷⁻³⁰. If PrP^{C} and PrP^{Sc} are treated with the proteolytic enzyme proteinase K, PrP^{C} is completely digested, whereas PrP^{Sc} is only partially digested on account of its structural dissimilarity. The proteinase K-resistant fragment PrP²⁷⁻³⁰ remains, which is then detected. The digestion by proteinase K requires additional work steps in these tests and exact controls of the concentration and time of action of the enzyme, so that after prolonged treatment the pathological prions may also be almost completely digested. Since the digestion first takes place in the sample and thereafter the prions are specifically detected, this method loses sensitivity. A further common feature: the tests can only be carried out post-mortem and require less than one gram of tissue from the brainstem, in which particularly many PrP^{Sc} molecules are accumulated. A drawback with all three tests is the insufficient sensitivity. The disease must be at an advanced stage with a correspondingly marked accumulation of BSE prions in order that clear test results can be obtained. Official authorities and institutes therefore employ other methods such as histopathology and immune histochemistry in suspected cases or to ensure a diagnosis. New techniques such as immune-PRC, specific ligand adsorption and fluorescence correlation spectroscopy (FCS) are being researched in order to improve the test sensitivity.

Another method, the conformation-dependent assay (conformation dependent immunoassay-CDI; Safar J. et al., Nature Medicine 1998, 4: 10, 1157-1165), is based on the specific conformation of the PrP^{Sc} molecule, and more precisely on the partially concealed binding site for monoclonal antibody 3F4. The ratio of the signal between native and denatured (unfolded PrP molecule) sample is used to detect the pathological form. This method also requires an additional preparatory treatment of the sample and is relatively time-consuming.

Another series of detection methods employs techniques for enriching the sample with pathological prions. One method of the series is the PMCA (protein misfolding cycle amplification) method from Soto (firm Serono; Castilla J. et al. Nature Medicine Online Publication 28.08.2005). For this, PrP^{Sc} is incubated in the excess of PrP^{C} in order to multiply the PrP^{Sc} aggregates which are destroyed in the subsequent ultrasound treatment, so that new, smaller aggregates are formed. The latter serve as a “matrix” for the formation of newer PrP^{Sc} aggregates.

The cycles are repeated many times (up to 150 times). In the PMCA method, at least 75 hours pass until the reported sensitivity is reached. In addition, hamster brain tissue is added as a “matrix” and it is unclear what stage of the infection the examined animals were in.

The serin protease plasmin (preferred cleavage site Lys-Xaa>Arg-Xaa) is an enzyme synthesised from plasminogen, an ubiquitary zymogen precursor, which plays an important role in the transformation of fibrin into soluble products (fibrinolysis) and in the proteolytic degradation of the extracellular matrix (plasma-induced proteolysis). It has recently been reported that plasmin is capable of cleaving PrP^{C} in vitro, and that PrP^{C} and the NH₂ region of the PrP molecule can stimulate t-PA (tissue-type plasminogen activator) imparted plasmin formation. It has also been found that the primary cleavage site of plasmin lies on the PrP molecule in the region of amino acid residues 108-112. No further findings are available, however, concerning the activity of plasmin with respect to the pathological form (PrP^{Sc}).

Hitherto, there has not therefore been any routinely usable test method with which an unequivocal early diagnosis can be made on a living animal or man during the incubation period, i.e. before the onset of clinically detectable symptoms.

There is therefore a need for a quick test for the detection of pathological prions, which overcomes the aforementioned drawbacks of the prior art.

The problem of the present invention, therefore, was to make available a test for the detection of pathological prions, which displays a high degree of sensitivity, which can be carried out with little time consumption and at comparatively low cost, if need be automatically, which is capable of detecting pathological prions at an early stage of the disease, and in which a proteinase K treatment can be dispensed with.

This and other problems, which are readily obvious to the person skilled in the art, are solved by the invention described below.

It was found, surprisingly, that the pathological form of the prion protein can be detected in vitro in a sample with a high degree of selectivity and at relatively low cost if:
a) capture antibodies, which recognise both the pathological (PrP^{Sc}) as well as the non-pathological form (PrP^{C}) of the prion protein, are fixed on a solid phase;
b) the sample is incubated with the fixed antibodies from step a), the pathological and the non-pathological form of the prion protein binding to the fixed antibodies thereby forming complexes;
c) the sample is separated from the complexes that have arisen;
d) the complexes are incubated with plasmin, whereby the non-pathological form of the prion protein is cleaved;
e) the cleaved fragments obtained through the incubation with plasmin are separated from the complexes; and
f) the un-cleaved prion protein contained in the complexes is detected with detection antibodies.

The non-pathological form of the prion protein is surprisingly cleaved by the treatment with plasmin, whereas the pathological form of the prion protein remains undigested. The pathological form has the same amino acid sequence as the physiological form of the prion protein, but a spatial structure differing therefrom. It has been found that the primary cleavage site for plasmin lies, in all the investigated species, in the region of amino acid residues 106 to 126 of the prion protein. The primary cleavage site of the pathological form lies partially concealed, camouflaged (“buried core”) and is thus difficultly accessible for the enzymatic activity of the plasmin. The good cleavability of the physiological form compared to the poor cleavability of the pathological form is the principal of the method developed here for distinguishing between the two prion forms.

According to the invention, pathological prion proteins are detected with this method.

The designations used therein denote the following:
PrP: the prion protein in general, for example when reference is made to structural characteristics of the prion protein;
PrP^{C}: the cellular form of the prion protein, i.e. its non-pathological form present in healthy cells; and
PrP^{Sc}: the pathological form of the prion protein.

The sample taken for the method can in principle originate from any human or animal subject suspected of presenting the pathological form of the prion protein. The sample can for example be of human origin, or originate from a cow or a hamster. The sample can be taken from a living or dead subject. In principle, any liquid or solid material originating from the subject's body that might contain the pathological form of the prion protein can serve as the starting material for the sample. Exemplary starting materials for the samples may be blood samples, tissue samples or body fluids such as urine, milk, cerebrospinal fluid or saliva. Especially in the case of solid samples it may be necessary first to decompose the starting material so that the pathological form of the prion protein is present in a suitable form for the method according to the invention. These decomposition processes have long been well known to the person skilled in the art.

The capture antibody is first fixed on a solid phase in the method according to the invention.

The capture antibody has the property of recognising and binding both the pathological form (PrP^{Sc}) as well as the non-pathological form of the prion protein (PrP^{C}). The capture antibodies can for example be monoclonal or polyclonal. Suitable capture antibodies can be self-produced according to standard methods or can be obtained commercially. Exemplary capture antibodies are the anti-PrP antibodies SAF32 and SAF61 (firm Spi-Bio, Montigny le Bretonneux, France).

As a solid phase, use can in principle be made of all solid materials which enable the fixing of the capture antibody, and which do not prevent the detection of the pathological form of the prion protein. As solid phases, use is preferably made of microtitre plates or magnetic or non-magnetic beads. The use of a microtitre plate as a solid phase is particularly preferred.

The fixing of the capture antibody to the solid phase can take place in any way known to the person skilled in the art. The capture antibody can be bound directly to the solid phase. For example, the capture antibody can be coupled covalently to the solid phase. On the other hand, the capture antibody can also be adsorbed at the surface of the solid phase. For this purpose, it is for example pipetted onto the bottom of a well of a microtitre plate and incubated for a suitable period (for example at least 16 hours) at a suitable temperature (for example 4° C.). It is also possible to couple the capture antibody with the solid phase by means of the biotin/avidin or streptavidin system known to the person skilled in the art. Alternatively, the fixing of the capture antibody can also take place by means of a bridging antibody, which imparts the binding of the capture antibody to the solid phase. It is preferable, however, for the capture antibody to be fixed directly to the solid phase.

After the fixing, free binding sites of the solid phase can be saturated with a suitable blocking buffer, the individual constituents whereof are known to the person skilled in the art. A suitable blocking buffer can for example comprise a suitable buffer system with a blocking reagent, such as for example bovine serum albumin (BSA).

After the fixing of the capture antibodies to the solid phase, the incubation with the sample takes place. The binding of the pathological (PrP^{Sc}) and the non-pathological form (PrP^{C}) of the prion protein to the fixed capture antibodies thereby takes place. The incubation takes place for a period which is sufficient for both forms of the prion protein to be bound as quantitatively as possible by the capture antibodies. The incubation time preferably amounts to not more than 2 hours.

After the incubation, the remaining sample is separated from the arising complexes comprising solid phase, capture antibodies and prion proteins. If a microtitre plate is used as a solid phase, the removal of the sample can take place for example by sucking off. If beads are used as a solid phase, the complexes containing the beads can be sedimented by centrifugation or, in the case of magnetic beads, by the effect of magnetic force, and the sample present in the supernatant can be removed.

The complexes comprising solid phase, capture antibodies and both forms of the prion protein are then mixed with plasmin. Underlying this step is the decisive and surprising principle that plasmin specifically cleaves the non-pathological form of the prion protein (PrP^{C}) contained in the complexes, but on the other hand does not cleave the pathological form of the prion protein (PrP^{Sc}) also contained in the complexes. After the cleaving of the non-pathological form of the prion protein, the complexes contain the solid phase, the capture antibodies and the intact, undigested pathological form of the prion protein (PrP^{Sc}), and respectively the cleavage fragment of the non-pathological form of the prion protein bound by the capture antibodies. The cleavage fragments of the non-pathological form of the prion protein that are not bound by the capture antibodies are removed from the complexes in this step.

The plasmin used for the cleaving of PrP^{C} is not further restricted, except that it must be in a position to cleave specifically the non-pathological form, but not the pathological form of the prion protein. Thus, for example, it can be recombinant or native plasmin. It can be produced in a manner known to the person skilled in the art, for example by activation of plasminogen on an activator (for example urokinase or streptokinase), which can for example be matrix-bound. It can be human plasmin or plasmin from other species. It is clear to the person skilled in the art that it is possible also to introduce mutations or deletions into the amino acid sequence of plasmin, without the inventive activity of plasmin thereby being adversely affected. Plasmin modified in this way is also covered by the present invention.

For the cleaving of the non-pathological form of the prion protein, plasmin is preferably present in a solution which contains a physiological buffer, such as for example PBS. The concentration of plasmin is selected such that it is sufficient for the cleaving of the non-pathological form of the prion protein contained in the complexes for the time interval provided for the cleavage. The concentration of plasmin preferably amounts to 10 nM to 2 μM, more preferably 25 nM to 1 μm, and still more preferably 40 nM to 60 nM. The incubation time of the complexes with plasmin is not particularly restricted. The incubation time preferably amounts, however, to not more than 30 minutes.

In a preferred embodiment, the cleaving of the non-pathological form of the prion protein is stopped by the addition of a suitable reagent which inhibits the activity of plasmin. Aprotinin is preferably used for this. The reagent inhibiting the activity of plasmin is added in solid or preferably liquid form and in a concentration sufficient for the inhibition of the plasmin activity. In the case of aprotinin, the preferred concentration amounts to 4 to 6 μM.

The cleavage fragments of the non-pathological form of the prion protein obtained by the incubation with plasmin that are not bound by the capture antibody are then separated from the complexes comprising solid phase, capture antibodies and the pathological form of the prion protein (PrP^{Sc}), and respectively the cleavage fragments of the non-pathological form of the prion protein (PrP^{C}) generated by the cleavage with plasmin and bound by the capture antibodies. The nature of the separation is adapted to the detection system used, in particular to the solid phase used. In all cases, it is preferable to remove the unbound PrP^{C} cleavage fragments simply by sucking off.

After the separation of the unbound PrP^{C} cleavage fragments from the complexes, the un-cleaved prion protein contained in the complexes is detected with detection antibodies. The un-cleaved prion protein is, essentially, exclusively the pathological form of the prion protein (PrP^{Sc}). The detection antibodies to be used for its detection have the capacity to bind PrP^{Sc} specifically. Since the non-pathological form of the prion protein has essentially been completely removed from the complexes, an antibody can also be used as a detection antibody that recognises both PrP^{Sc} and PrP^{C}.

The detection of the detection antibody takes place in any way known to the person skilled in the art. A large number of suitable detection methods are known from the prior art for this purpose. As a non-exhaustive selection of these techniques, mention may be made of ELISA (enzyme-linked immunosorbent assay), EIA (enzyme-linked immunoassay), nanobead technology (for example with nanobeads marked with europium), fluorescence (for example, time-resolved fluorescence) and luminescence methods.

The detection preferably takes place by means of ELISA (enzyme-linked immunoabsorbent assay) techniques known to the person skilled in the art. For example, the detection antibody can be conjugated with biotin and its detection can take place via a streptavidin polyperoxidase conjugate, which is mixed immediately before the measurement with activators such as for example luminal or TMB (3,3′,5,5′-tetraethyl benzidine). The detection preferably takes place by means of the biotin/streptavidin or avidin system when the capture antibody has not already been fixed to the solid phase with this system. Examples of detection antibodies according to the invention are biotinylated anti-PrP antibodies SAF32 and SAF61 (firm Spi-Bio, Montigny le Bretonneux, France).

It is therefore clear to the person skilled in the art that the detection antibodies can be conjugated with a detection molecule, a group suitable for the detection or also with solid structures (for example microbeads or nanobeads, such as for example europium nanobeads), in order to enable the detection with one of the detection methods mentioned above or others known from the prior art. It may therefore be preferable, for example, to use detection antibodies which are conjugated with biotin or fluorescence markers (such as for example forescein-isothiocyanate or rhodamin). The detection antibodies can for example be polyclonal or preferably monoclonal. Suitable detection antibodies can be self-produced according to standard methods or can be obtained commercially.

In a preferred embodiment, the capture antibody and the detection antibody are selected such that the capture antibody is directed to an epitope of the prion protein which lies aminoterminally with respect to the primary cleavage site of plasmin, if the detection antibody recognises an epitope of the prion protein which is arranged carboxyterminally with respect to the primary cleavage site of plasmin. Accordingly, it is also preferable for the capture antibody to be directed to an epitope of the prion protein which lies carboxyterminally with respect to the primary cleavage site of plasmin, if the detection antibody recognises an epitope of the prion protein which is arranged aminoterminally with respect to the primary cleavage site of plasmin.

In a preferred embodiment, therefore, detection antibody and capture antibody are selected such that the capture antibody is directed to an epitope of the prion protein which is located in the region of amino acid residues 1-110, if the detection antibody is directed to an epitope which lies outside this region, or the detection antibody is directed to an epitope which is located in the region of amino acid residues 1-110, if the capture antibody is directed to an epitope which lies outside this region.

In another preferred embodiment, the detection of the un-cleaved prion protein contained in the complexes takes place quantitatively. This is possible, for example, if an ELISA test is used for the detection of the prion protein, in which the measured signal intensity is proportional to the quantity of the detected prion protein in the sample. If the method according to the invention is used for example on duplicates of samples, and if the one sample is incubated with a quantity of plasmin sufficient for the complete cleavage of the non-pathological form of the prion protein for a time which is sufficient for this (for example for 30 minutes with 50 nM plasmin) and if the other sample is left untreated, then it is possible, on the basis of the ratio of the signal intensity of the treated sample to the signal intensity of the untreated sample, to ascertain the extent to which the cleavage of the overall population of the prion protein has taken place.

Moreover, it may be advantageous for the method according to the invention if, between one or more individual steps of the method, one or more washing steps are carried out, for which suitable washing buffers known to the person skilled in the art are used. Physiological buffer solutions are preferably used for this, such as PBS or TBS, which can be supplemented with detergents such as Tween-20.

According to the invention, kits can also be used for performing the method according to the invention for detecting pathological prion proteins.

The kits according to the invention contain capture antibodies, which are directed to both the pathological (PrP^{Sc}) and also the non-pathological form (PrP^{C}) of the prion protein, plasmin and detection antibodies.

The features of these components of the kit essential to the invention have already been described in detail.

Thus, it may be advantageous, for example, for the capture antibodies to be present already fixed on a solid phase. Microtitre plates or magnetic or non-magnetic beads are preferably used as a solid phase.

In one embodiment, the detection antibodies contained in the kit recognise both the pathological and the non-pathological form of the prion protein.

In a preferred embodiment, the capture antibody contained in the kit is directed to an epitope of the prion protein which lies aminoterminally with respect to the primary cleavage site of plasmin, if the detection antibody recognises an epitope of the prion protein which is arranged carboxyterminally with respect to the primary cleavage site of plasmin. Alternatively, it may also be preferable for the capture antibody to be directed to an epitope of the prion protein which lies carboxyterminally with respect to the primary cleavage site of plasmin, if the detection antibody recognises an epitope of the prion protein which is arranged aminoterminally with respect to the primary cleavage site of plasmin.

In another preferred embodiment, the capture antibody contained in the kit is directed to an epitope of the prion protein which is located in the region of amino acid residues 1-110, if the detection antibody is directed to an epitope which lies outside this region. On the other hand, the detection antibody contained in the kit can also be directed to an epitope which is located in the region of amino acid residues 1-110, if the capture antibody is directed to an epitope which lies outside this region.

In a still further preferred embodiment, the kit additionally contains a blocking buffer for the saturation of three binding sites of the solid phase, a washing buffer and/or aprotinin.

In a still further embodiment, plasmin is present in the kit either dissolved in a buffer solution or lyophilised as a solid.

If aprotinin is contained in the kit, this can also be present dissolved in a buffer solution or lyophilised as a solid. Any additions to the solutions contained in the kit (for example detergents, blocking reagents) can also be contained in the kit.

The method according to the invention makes it possible to detect pathological prion proteins at an early stage of disease at low cost and with little time consumption with high specificity and sensitivity, if need be as part of an automated test. The detection of PrP^{Sc} is successful in different species, such as man, hamster or cow, with extraordinarily high sensitivity.

By determining the ID₅₀ dose (the infectious dose which causes disease in at least 50% of the exposed animals; Prusiner S., Proc. Natl. Acad. Sci. USA, 1998, 10, 95, 13363-13383), the sensitivity of tests for the detection of pathological prions can be ascertained (the lower the ID₅₀/ml value, the higher the sensitivity of the test). Values of less than 1000 ID₅₀/ml can be achieved with the method according to the invention. The ID₅₀/ml values of the commercially available tests Prionics Check (ID₅₀/ml: 1,000,000-100,000; BSE homogenate as sample; detection limit 100-10⁻¹ dilution), Platelia® BSE test (ID₅₀/ml: 3,000; BSE homogenate as sample; detection limit 10^{-2.5} dilution) and Enfer TSA (ID₅₀/ml: 30,000; BSE homogenate as sample; detection limit 10^{-1.5} dilution) are quoted for purposes of comparison.

The method according to the invention is based on the good cleavability of the physiological PrP form by plasmin compared to the poor cleavability of the pathological form. The specific folding of the PrP^{Sc} molecule which conceals the primary cleavage site for plasmin and the higher enzymatic selectivity of the plasmin permit a distinction to be made between the two forms after the immobilisation of the prions. The use of plasmin has the advantage over proteinase K that only PrP^{C} is cleaved and not completely digested, the employed antibodies remaining intact.

Overall, the method takes approximately 3.5 hours and does not require any special preliminary treatment of the sample, as for example the Platelia® BSE test. Unlike the Enfer test, where the adsorption of the prions takes place non-specifically on the surface of the microtitre plate, the binding of the prions is specific from the outset. Furthermore, the time consumption with the method according to the invention is much smaller than in the case of the Prionics Check, in which the detection takes place according to a Western blot.

The method according to the invention reduces the dependence on a specific antibody type and is thus extremely flexible: since PrP^{C} is basically cleaved by plasmin into two fragments, different antibodies can be used, so that either the aminoterminal region or the carboxyterminal region of the PrP molecule can be detected.

In contrast with all the other methods known previously, moreover, the method according to the invention permits the measurement of the initial rate of the PrP^{C} cleavage. The method according to the invention can easily be carried out in an automated manner, which is favourable for routine use. In addition, the method could also be used to increase the sensitivity of other immunological methods for the detection of prion (“mild” digestion, thus better signal-to-noise ratio).

The present invention is illustrated below with the aid of examples, but the latter should not be understood to be limiting.

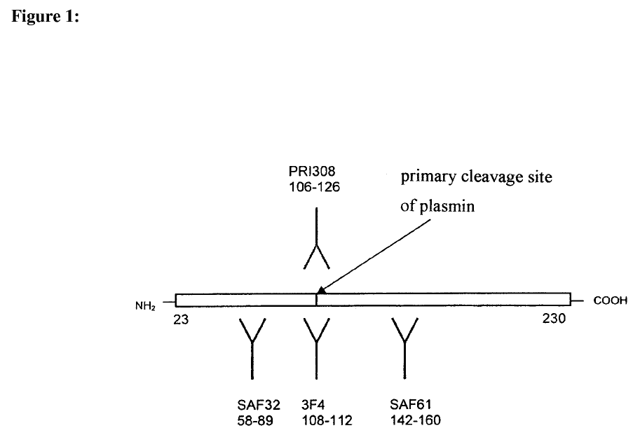

FIG. 1 shows the epitope of the anti-PrP antibody used for the exemplary tests.

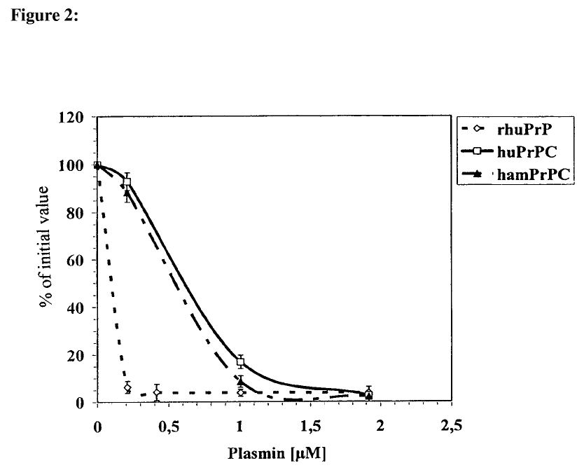

FIG. 2 shows the in vitro cleavage of non-immobilised prion proteins by human plasmin as a function of different plasmin concentrations. In the legend, the designations signify the following: rhuPrP: recombinant human PrP, huPrP^{C}: human PrP^{C} (serum), hamPrP^{C}: hamster PrP^{C} (brain homogenate). Mean values and standard deviations of three independent tests are represented. The percentage ratio of the intensity of a sample after the stated incubation time with plasmin to the intensity of the sample without incubation with plasmin is shown. All the indicated values are background-corrected.

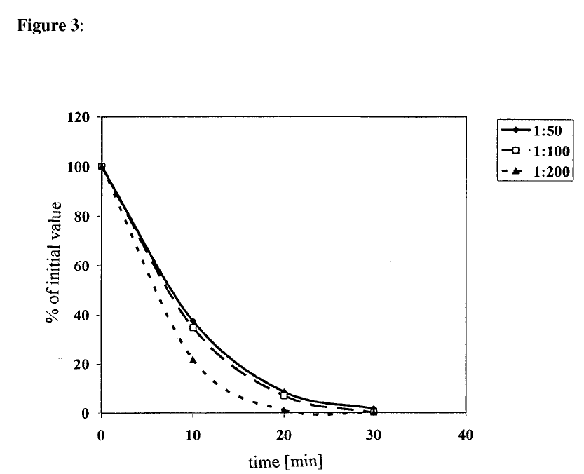

FIG. 3 shows the cleavage of native human PrP^{C} by human plasmin after immobilisation on the microtitre plate. SAF32 (recognises epitope between amino acid residues 58 and 89 of the PrP molecule) was used as a capture antibody and biotinylated 3F4 (recognises epitope between amino acid residues 108 and 111 of the PrP molecule) was used as a detection antibody. Various sample dilutions were treated directly on the plate with plasmin with different incubation times at 37° C. The percentage ratio of the intensity of a sample after the stated incubation time with plasmin to the intensity of the sample without incubation with plasmin is shown. All the indicated values are background-corrected.

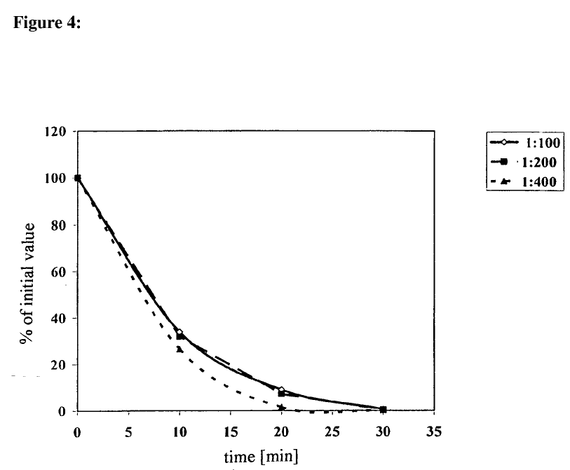

FIG. 4 shows the cleavage of native hamster PrP^{C} by human plasmin after immobilisation on the microtitre plate. SAF32 (recognises epitope between amino acid residues 58 and 89 of the PrP molecule) was used as a capture antibody and biotinylated 3F4 (recognises epitope between amino acid residues 108 and 111 of the PrP molecule) was used as a detection antibody. Various sample dilutions were treated directly on the plate with plasmin with different incubation times at 37° C. The percentage ratio of the intensity of a sample after the stated incubation time with plasmin to the intensity of the sample without incubation with plasmin is shown.

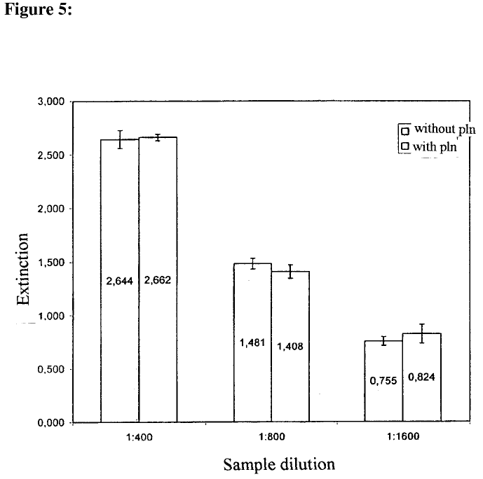

FIG. 5 shows the cleavage of native PrP^{C} (from hamster brain homogenate) by human plasmin after immobilisation on the microtitre plate. PRI3O8 (recognises epitope between amino acid residues 106-126 of the PrP molecule) was used as a capture antibody and biotinylated SAF32 (recognises epitope between amino acid residues 58-89 of the PrP molecule) was used as a detection antibody. The PRI3O8 epitope contains the cleavage site of the plasmin, as a result of which the cleavage of PrP^{C} is suppressed.

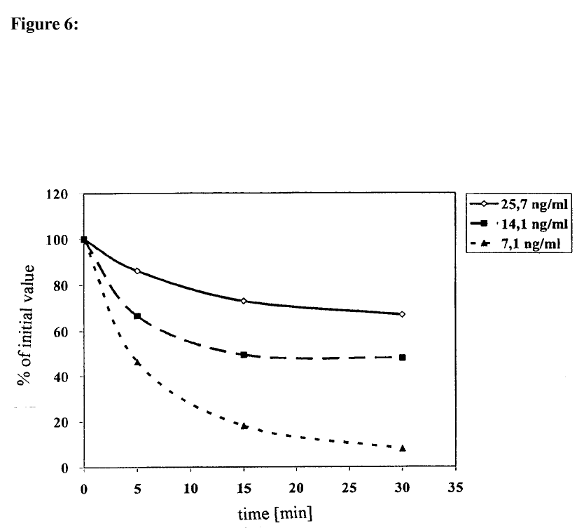

FIG. 6 shows the cleavage of recombinant human PrP with replaced lysine residues in lysine cluster 2 (dLC2) by human plasmin after immobilisation on the microtitre plate. Lysine cluster 2 comprises amino acid residues 101 to 110 of PrP. The lysine residues contained therein at position 101, 104, 106 and 110 were replaced by alanine. Different sample concentrations were investigated. SAF61 (recognises epitope between amino acid residues 142-160 of the PrP molecule) was used as a capture antibody and biotinylated SAF32 (recognises epitope between amino acid residues 58-89 of the PrP molecule) was used as a detection antibody. The percentage ratio of the intensity of a sample after the stated incubation time with plasmin to the intensity of the sample without incubation with plasmin is shown. All the indicated values are background-corrected.

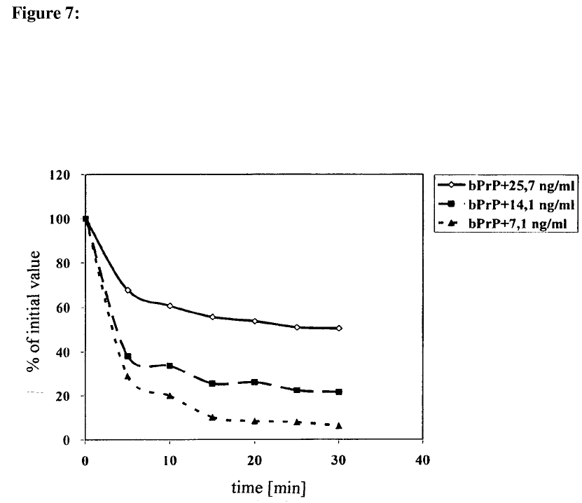

FIG. 7 shows the cleavage of recombinant human PrP with replaced lysine residues in lysine cluster 2 (dLC2) by human plasmin after immobilisation on the microtitre plate in the excess of bPrP (bovine PrP from bovine brain homogenate). SAF61 (recognises epitope between amino acid residues 142-160 of the PrP molecule) was used as a capture antibody and biotinylated SAF32 (recognises epitope between amino acid residues 58-89 of the PrP molecule) was used as a detection antibody. The percentage ratio of the intensity of a sample after the stated incubation time with plasmin to the intensity of the sample without incubation with plasmin is shown. All the indicated values are background-corrected.

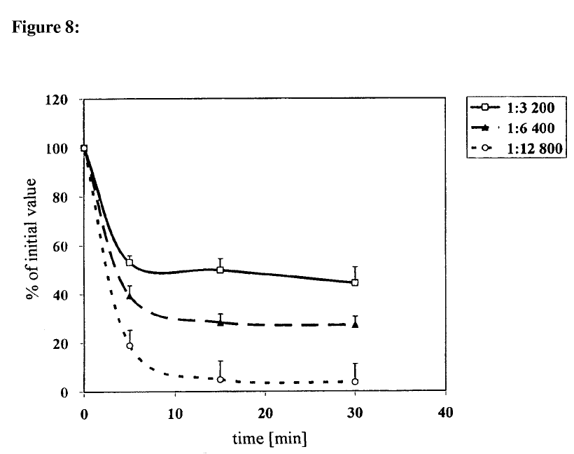

FIG. 8 shows the cleavage of PrP^{Sc} from hamster brain homogenate by human plasmin after immobilisation on a microtitre plate. SAF61 (recognises epitope between amino acid residues 142-160 of the PrP molecule) was used as a capture antibody and SAF32-biotin (recognises epitope between amino acid residues 58-89 of the PrP molecule) was used as a detection antibody. Various dilutions of brain homogenates of animals infected with scrapie are represented. The percentage ratio of the intensity of a sample after the stated incubation time with plasmin to the intensity of the sample without incubation with plasmin is shown. All the indicated values are background-corrected. Mean values and standard deviations from three independent tests are represented.

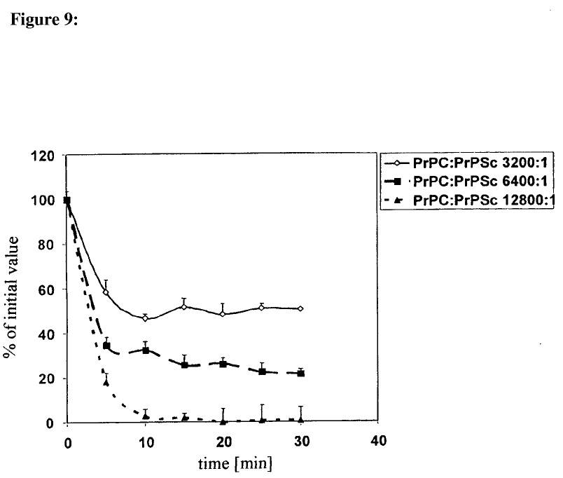

FIG. 9: Cleavage of PrP^{Sc} (from hamster brain homogenate) in the excess of native PrP^{C} by human plasmin after immobilisation on a microtitre plate. SAF61 (recognises epitope between amino acid residues 142-160 of the PrP molecule) was used as a capture antibody and biotinylated SAF32 (recognises epitope between amino acid residues 58-89 of the PrP molecule) was used as a detection antibody. PrP^{C} denotes the normal, cellular form of PrP and PrP^{Sc} the pathological form of PrP. The brain homogenate from scrapie hamster was diluted in brain homogenate from healthy hamsters. Mean values and standard deviations from three independent tests are represented. The percentage ratio of the intensity of a sample after the stated incubation time with plasmin to the intensity of the sample without incubation with plasmin is shown. All the indicated values are background-corrected.

### EXAMPLE 1

### Cleavage of Non-Immobilised PrP by Plasmin

Both the recombinant human PrP (rhuPrP), as well as native PrP^{C} (human serum, hamster brain homogenate) were cleaved in vitro with plasmin in a series of tests, the plasmin digestion however taking place in a sample tube, and the prion proteins were not immobilised at the time of digestion.

Recombinant human PrP (rhyPrP), human PrP^{C} (serum; huPrP^{C}) and hamster PrP^{C} (brain homogenate; hamPrP^{C}) were incubated in initial concentrations of 553 to 575 pg/ml (calibration against recombinant human PrP; firm Roboscreen) for 30 minutes at 37° C. with different concentrations on human plasmin in a sample tube. The reaction was then stopped by the addition of aprotinin and PrP was detected by an ELISA test. SAF32 (recognises epitope between amino acid residues 58 and 89 of the PrP molecule) was used as a capture antibody and 3F4 coupled with biotin (recognises epitope between amino acid residues 108 and 111 of the PrP molecule) was used as a detection antibody. For the ELISA test, the sample was mixed with a streptavidin polyperoxidase conjugate (SApolyHRP, firm Pierce, Rockford, USA), which was diluted 1:5000 in reaction buffer (1 part blocking buffer+4 parts PBS), and incubated for 20 minutes at room temperature. TMB (3,3′,5,5′-tetramethyl benzidine) was then added to the sample as a substrate and incubated for 30 minutes. After the addition of the stopping solution (0.25% H₂SO₄ in distilled water), the extinction of the sample was measured at 405 nm using an ELISA reader (Tecan Genios; Tecan, Switzerland).

The epitope of the detection antibody lies precisely in the primary cleavage site of the prion protein, as a result of which only un-cleaved prion proteins in the sample can be detected by ELISA (FIG. 1).

It emerged that the cleavage of native PrP^{C} in vitro, through the presence of plasmin inhibitors present in the sample, can only be controlled with difficulty if the digestion is carried out in a sample tube without prior immobilisation of the prion proteins. A plasmin concentration of approximately 200 nM is therefore required for a significant cleavage of recombinant human PrP in the sample tube, whereas the same degree of cleavage for human PrP^{C} and hamster PrP^{C} is only reached with a plasmin concentration of over 1 μm (FIG. 2).

For this reason, a test was developed in which prions contained in a sample are first immobilised by means of a monoclonal antibody, then treated with human plasmin and the remaining, un-cleaved prions are then detected with another marked antibody. Since the sample is separated from the complexes of fixed antibodies and prion proteins before the plasmin digestion, plasmin inhibitors or plasmin substrates contained in the sample cannot inhibit or influence the plasmin activity during digestion.

### EXAMPLE 2

### Cleavage of Immobilised PrP by Plasmin

In further tests, the cleavage of PrP by plasmin was investigated after immobilisation by means of antibodies fixed on microtitre plates (FIG. 3 and FIG. 4). For this purpose, use was made both of antibodies which are directed to different epitopes of the PrP molecule (FIG. 1; FIG. 5), as well as rhuPrP, in which the lysine residues in the cleavage region of the plasmin are replaced by alanine (FIG. 6 and FIG. 7).

### EXAMPLE 2.1

### Cleavage of Immobilised PrP^{C} by Human Plasma or Hamster Brain Homogenate

For the cleavage of immobilised PrP^{C} from human plasma, three transparent microtitre plates (Lumi-Nunc Maxi-Sorp F96; firm Nunc, Wiesbaden) were coated with 100 μl/well of monoclonal antibody (Anti-PrP: SAF32, firm Spi-Bio, Montigny le Bretonneux, France; concentration: 1 μg/ml in carbonate/bicarbonate buffer pH 9.0, firm Perbio, Bonn) for 16-18 hours at 4° C. The residual fluid was sucked off and the free binding sites were saturated by the addition of 100 μl blocking buffer (Superblock, firm Perbio, Bonn) into each well for 1 hour. The blocking buffer was sucked off from the plates and the samples (pooled human citrate plasma, suitably diluted in reaction buffer: 1 part blocking buffer+4 parts PBS) were pipetted onto the plates. After two hours' incubation at room temperature, the plates were washed three times with washing buffer (TBS (Burph TBS, firm Pierce, Rockford, USA) with 0.5% Tween-20 (Surfact-Amps, firm Pierce, Rockford, USA)). 100 μl of 50 nM human plasmin (firm Chromogenix, Stockholm, Sweden) in PBS (firm Perbio, Bonn) was then pipetted into the wells. The plates were incubated for 0, 10, 20 and 30 minutes at 37° C. in a thermoshaker (THERMOSTAR, firm BMG, Offenburg) at 500 revolutions per minute. 25 μl of 5 μM aprotinin (firm Merck Biosciences, Schwalbach) in PBS was then pipetted into each well. After five minutes' incubation at room temperature, the plates were washed three times with the washing buffer. 100 μl of biotinylated detection antibody (3F4, firm Signet, Dedham, USA; 125 ng/ml) in reaction buffer was pipetted into each well for the detection of undigested PrP.

### ELISA Test:

For the ELISA test, incubation was carried out with the detection antibody for 1 hour at room temperature in the presence of gentle shaking in the dark. After washing six times with in each case 300 μl washing buffer (TBS (Burph TBS, firm Pierce, Rockford, USA) with 0.5% Tween-20 (Surfact-Amps, firm Pierce, Rockford, USA)), a streptavidin polyperoxidase conjugate (SApolyHRP, firm Pierce, Rockford, USA) was diluted 1:5000 in reaction buffer (1 part blocking buffer+4 parts PBS) and pipetted 100 μl per well onto the microtitre plates. After 20 minutes' incubation at room temperature in the presence of gentle shaking in the dark, washing was again carried out six times with in each case 300 μl washing buffer. 100 μl of TMB (3,3′,5,5′-tetramethyl benzidine) brought to room temperature was then pipetted in each case as a substrate into each well and incubated for 30 minutes at room temperature in the presence of moderate shaking in the dark. After the addition of 50 μl stopping solution (0.25% H₂SO₄ in distilled water) per well, the extinction of the sample was measured at 405 nm with an ELISA reader (Tecan Genios; Tecan, Switzerland).

The results of the cleavage of immobilised PrP^{C} from human plasma are shown in FIG. 3. Analogous to this, the cleavage of immobilised PrP^{C} from hamster brain homogenate was also carried out, with the sole exception that use was not made of pooled human citrate plasma for the sample, but rather of hamster brain homogenate from healthy animals. The results of the cleavage of immobilised PrP^{C} from hamster brain homogenate are shown in FIG. 4.

The exponentially diminishing percentage intensity, which can be seen in FIGS. 3 and 4, makes it clear that the biotinylated 3F4 used as a detection antibody, which recognises an epitope between amino acid residues 108 and 111 of the PrP molecule, detects epitopes less and less with advancing incubation time. This shows that the epitope of this PrP antibody lies on the PrP^{C} fragment, which was degraded by the cleavage with plasmin and washed out before the detection with the detection antibody.

It can also be seen that, after only 20 minutes' incubation with plasmin, the total quantity of immobilised PrP_{C} was cleaved by plasmin from human plasma of 1:200 dilution. With 1:50 and 1:100 dilution, almost no further un-cleaved human PrP^{C} was present after 30 minutes' plasmin incubation (FIG. 3).

In the case of hamster homogenate (1:400 dilution), the total quantity of immobilised PrP^{C} was cleaved by plasmin after 20 minutes' plasmin incubation. With 1:100 and 1:200 dilution, almost no further un-cleaved human PrP^{C} could be detected after 30 minutes' incubation with plasmin (FIG. 4).

### EXAMPLE 2.2

### Suppression of the Cleavage of PrP

In order to suppress the PrP cleavage by plasmin, two transparent microtitre plates (Lumi-Nunc Maxi-Sorp F96; firm Nunc, Wiesbaden) were coated with 100 μl/well of monoclonal antibody (Anti-PrP: PRI3O8, firm Spi-Bio, Montigny le Bretonneux, France, concentration: 1 μg/ml in carbonate/bicarbonate buffer pH 9.0 (firm Perbio, Bonn) for 16-18 hours at 4° C. The residual fluid was sucked off and the free binding sites were saturated by the addition of 100 μl blocking buffer (Superblock, firm Perbio, Bonn) into each well for 1 hour. The blocking buffer was sucked off from the plates and the samples (hamster brain homogenate extract from healthy animals, suitably diluted in reaction buffer: 1 part blocking buffer+4 parts PBS) were pipetted onto the plates. After two hours' incubation at room temperature, the plates were washed three times with washing buffer (TBS (Burph TBS, firm Pierce, Rockford, USA) with 0.5% Tween-20 (Surfact-Amps, firm Pierce, Rockford, USA). 100 μl of 50 nM human plasmin (firm Chromogenix, Stockholm, Sweden) in PBS (firm Perbio, Bonn) was pipetted onto one microtitre plate, whilst 100 μl of PBS was pipetted onto the second microtitre plate. The plates were incubated for 30 minutes at 37° C. in a thermoshaker (THERMOSTAR, firm BMG, Offenburg) at 500 revolutions per minute. 25 μl/well of 5 μM aprotinin (firm Merck Biosciences, Schwalbach) in PBS was then pipetted onto both plates. After 5 minutes' incubation at room temperature, the plates were washed three times with the washing buffer. 100 μl of biotinylated detection antibody (SAF32, firm Spi-Bio, Montigny le Bretonneux, France; 125 ng/ml) in reaction buffer was pipetted into each well for the detection of undigested PrP. The ELISA test was then carried out according to the above instructions.

As can be seen from FIG. 5, cleavage with plasmin is not possible after the immobilisation of native PrP^{C} by means of a capture antibody (PRI3O8) which recognises amino acid residues 106-126 of the PrP molecule as an epitope. This shows that the primary cleavage site of PrP^{C} is masked for plasmin by the binding of PrP^{C} by the capture antibody, and it must be located between amino acid residues 106-126 of the PrP molecule.

### EXAMPLE 2.3

### Cleavage of Recombinant PrP^{C}

For the cleavage of recombinant PrP^{C}, four transparent microtitre plates (Lumi-Nunc Maxi-Sorp F96; firm Nunc, Wiesbaden) were coated with 100 μl/well of monoclonal antibody (Anti-PrP: SAF61, firm Spi-Bio, Montigny le Bretonneux, France, concentration: 1 μg/ml in carbonate/bicarbonate buffer pH 9.0, firm Perbio, Bonn) for 16-18 hours at 4° C. The residual fluid was sucked off and the free binding sites were saturated by the addition of 100 μl blocking buffer (Superblock, firm Perbio, Bonn) into each well for 1 hour. The blocking buffer was sucked off from the plates and the samples (recombinant human PrP with lysine residues replaced by alanine in lysine cluster 2 (dLC2) from the Institut für Labormedizin, Charité, Campus Virchow Klinikum; suitably diluted in reaction buffer: 1 part blocking buffer+4 parts PBS) were pipetted onto the plates. After two hours' incubation at room temperature, the plates were washed three times with washing buffer (TBS (Burph TBS, firm Pierce, Rockford, USA) with 0.5% Tween-20 (Surfact-Amps, firm Pierce, Rockford, USA). 100 μl of 50 nM human plasmin (firm Chromogenix, Stockholm, Sweden) in PBS (firm Perbio, Bonn) was then pipetted into the microtitre plates. The plates were incubated for 0, 5, 15 and 30 minutes at 37° C. in a thermoshaker (THERMOSTAR, firm BMG, Offenburg) at 500 revolutions per minute. 25 μl/well of 5 μM aprotinin (firm Merck Biosciences, Schwalbach) in PBS was then pipetted onto all the plates. After 5 minutes' incubation at room temperature, the plates were washed three times with the washing buffer. 100 μl of biotinylated detection antibody (SAF32, firm Spi-Bio, Montigny le Bretonneux, France, 125 ng/ml) in reaction buffer was pipetted into each well for the detection of undigested PrP^{C}. The ELISA test was then carried out according to the above instructions.

The results (FIG. 6) show that the replacement of lysine residues in lysine cluster 2 (dLC2) of human PrP by alanine leads to a significant impairment of the cleavage of PrP by plasmin. After 30 minutes, over 70% of mutated PrP^{C} is still undigested in the sample which contains the mutated PrP^{C} in a concentration of 25.7 ng/ml. The plasmin cleavage is also greatly inhibited at lower concentrations of the mutated PrP^{C}.

### EXAMPLE 2.4

### Cleavage of Immobilised Recombinant and Native PrP^{C} in a Mixture

For the cleavage of immobilised recombinant and native PrP^{C} in a mixture, seven transparent microtitre plates (Lumi-Nunc Maxi-Sorp F96; firm Nunc, Wiesbaden) were coated with 100 μl/well of monoclonal antibody (Anti-PrP: SAF61, firm Spi-Bio, Montigny le Bretonneux, France, concentration: 1 μg/ml in carbonate/bicarbonate buffer pH 9.0, firm Perbio, Bonn) for 16-18 hours at 4° C. The residual fluid was sucked off and the free binding sites were saturated by the addition of 100 μl blocking buffer (Superblock, firm Perbio, Bonn) into each well for 1 hour. The blocking buffer was sucked off from the plates and the samples (recombinant human PrP with lysine residues replaced by alanine in lysine cluster 2 (dLC2) from the Institut für Labormedizin, Charité, Campus Virchow Klinikum; diluted to 25.7; 14.1 and 7.1 ng/ml in bovine brain homogenate, 1:100 diluted in reaction buffer; reaction buffer: 1 part blocking buffer+4 parts PBS) were pipetted onto the plates. After two hours' incubation at room temperature, the plates were washed three times with washing buffer (TBS (Burph TBS, firm Pierce, Rockford, USA) with 0.5% Tween-20 (Surfact-Amps, firm Pierce, Rockford, USA)). 100 μl of 50 nM human plasmin (firm Chromogenix, Stockholm, Sweden) in PBS (firm Perbio, Bonn) was pipetted into the microtitre plates. The plates were incubated for 0, 5, 10, 15, 20, 25 and 30 minutes at 37° C. in a thermoshaker (THERMOSTAR, firm BMG, Offenburg) at 500 revolutions per minute. 25 μl/well of 5 μM aprotinin (firm Merck Biosciences, Schwalbach) in PBS was then pipetted onto all the plates. After 5 minutes' incubation at room temperature, the plates were washed three times with the washing buffer. 100 μl of biotinylated detection antibody (SAF32, firm Spi-Bio, Montigny le Bretonneux, France, 125 ng/ml) in reaction buffer was pipetted into each well for the detection of undigested PrP^{C}. The ELISA test was then carried out according to the above instructions.

It can be seen from FIG. 7 that native bovine PrP^{C} can also be cleaved from a mixture with different concentrations of mutated, recombinant human PrP, the cleavage whereof by plasmin is strongly suppressed on account of the mutation (see previous example).

### EXAMPLE 3

### Test for Detecting PrP^{Sc} in a Sample with PrP^{C}

In a further series of tests, scrapie samples (hamster 263K strain) were investigated with plasmin. Brain homogenate from infected animals in the terminal stage was investigated in various concentrations for the presence of PrP^{Sc} (FIG. 8). Moreover, various quantities of brain homogenate from infected animals were added to brain homogenate from healthy animals, in order to check the detection of the pathological form in the presence of larger concentrations of the normal form (FIG. 9).

### EXAMPLE 3.1

### Cleavage of PrP^{C} from Hamster Brain Homogenate from Infected Animals by Plasmin After Immobilisation on a Microtitre Plate

For the preparation of the samples, 40 μl of hamster brain homogenate from animals infected with scrapie was mixed with 40 μl of 2% sarkosyl (firm Sigman, Seelze), treated for 60 seconds in stage 3 with ultrasound (ultrasound device UP100H, firm Dr. Hielscher, Teltow) and incubated for 2 hours at room temperature on a rotor (neoLab-Rotor, firm Roth, Karlsruhe). A dilution series was then prepared in reaction buffer.

Four transparent microtitre plates (Lumi-Nunc Maxi-Sorp F96; firm Nunc, Wiesbaden) were coated with 100 μl/well of monoclonal antibody (Anti-PrP: SAF61, firm Spi-Bio, Montigny le Bretonneux, France, concentration: 1 μg/ml in carbonate/bicarbonate buffer pH 9.0, firm Perbio, Bonn) for 16-18 hours at 4° C. The residual fluid was sucked off and the free binding sites were saturated by the addition of 100 μl blocking buffer (Superblock, firm Perbio, Bonn) into each well for 1 hour. The blocking buffer was sucked off from the plates and the prepared samples were pipetted onto the plates. After two hours' incubation at room temperature, the plates were washed three times with washing buffer (TBS (Burph TBS, firm Pierce, Rockford, USA) with 0.5% Tween-20 (Surfact-Amps, firm Pierce, Rockford, USA)). 100 μl of 50 nM human plasmin (firm Chromogenix, Stockholm, Sweden) in PBS (firm Perbio, Bonn) was pipetted into the microtitre plates. The plates were incubated for 5, 15 and 30 minutes at 37° C. in a thermoshaker (THERMOSTAR, firm BMG, Offenburg) at 500 revolutions per minute. 25 μl/well of 5 μM aprotinin (firm Merck Biosciences, Schwalbach) in PBS was then pipetted onto all the plates. After 5 minutes' incubation at room temperature, the plates were washed three times with the washing buffer. 100 μl of biotinylated detection antibody (SAF32, firm Spi-Bio, Montigny le Bretonneux, France, 125 ng/ml) in reaction buffer was pipetted into each well for the detection of undigested PrP molecules. The ELISA test was then carried out according to the above instructions.

It emerged that PrP^{Sc} itself can still be clearly detected in buffer in a 1:6400 dilution of the hamster brain homogenate from animals infected with scrapie. The measured intensity is proportional to the concentration of the hamster brain homogenate (and thus of the PrP^{Sc}).

### EXAMPLE 3.2

### Cleavage of PrP^{Sc} from Hamster Brain Homogenate in the Excess of PrP^{C} by Plasmin After Immobilisation on a Microtitre Plate

For the preparation of the samples, 40 μl of hamster brain homogenate from animals infected with scrapie and from healthy animals was mixed with 40 μl of 2% sarkosyl (firm Sigman, Seelze), treated for 60 seconds in stage 3 with ultrasound (ultrasound device UP100H, firm Dr. Hielscher, Teltow) and incubated for 2 hours at room temperature on a rotor (neoLab-Rotor, firm Roth, Karlsruhe). A dilution series of scrapie homogenates was then prepared in normal homogenates. The samples were diluted 1:100 in reaction buffer immediately before the pipetting onto the plates.

Seven transparent microtitre plates (Lumi-Nunc Maxi-Sorp F96; firm Nunc, Wiesbaden) were coated with 100 μl/well of monoclonal antibody (Anti-PrP: SAF61, firm Spi-Bio, Montigny le Bretonneux, France, concentration: 1 μg/ml in carbonate/bicarbonate buffer pH 9.0, firm Perbio, Bonn) for 16-18 hours at 4° C. The residual fluid was sucked off and the free binding sites were saturated by the addition of 100 μl blocking buffer (Superblock, firm Perbio, Bonn) into each well for 1 hour. The blocking buffer was sucked off from the plates and the prepared samples were pipetted onto the plates. After two hours' incubation at room temperature, the plates were washed three times with washing buffer (TBS (Burph TBS, firm Pierce, Rockford, USA) with 0.5% Tween-20 (Surfact-Amps, firm Pierce, Rockford, USA)). 100 μl of 50 nM human plasmin (firm Chromogenix, Stockholm, Sweden) in PBS (firm Perbio, Bonn) was pipetted into the microtitre plates. The plates were incubated for 5, 15 and 30 minutes at 37° C. in a thermoshaker (THERMOSTAR, firm BMG, Offenburg) at 500 revolutions per minute. 25 μl/well of 5 μM aprotinin (firm Merck Biosciences, Schwalbach) in PBS was then pipetted onto all the plates. After 5 minutes' incubation at room temperature, the plates were washed three times with the washing buffer. 100 μl of biotinylated detection antibody (SAF32, firm Spi-Bio, Montigny le Bretonneux, France, 125 ng/ml) in reaction buffer was pipetted into each well for the detection of undigested PrP molecules. The ELISA test was then carried out according to the above instructions.

It can be seen from FIG. 9 that the pathological form of the prion protein (PrP^{Sc}) can still be clearly detected even with a 6400-fold excess of the non-pathological form of the prion protein (PrP^{C}). This shows that the detection of PrP^{Sc} by the method according to the invention is successful not only on subjects in the terminal stage of the disease, in which the concentration of PrP^{Sc} exceeds the concentration of PrP^{C}, but is already possible well ahead of this, at an early pre-clinical stage.

1. A method for the detection of pathological prion proteins in vitro in a sample, comprising:
a) fixing capture antibodies, which recognise both the pathological (PrP^{Sc}) as well as the non-pathological form (PrP^{C}) of the prion protein, on a solid phase;
b) incubating the sample with the capture antibodies from step a), the pathological and the non-pathological form of the prion protein binding to the capture antibodies thereby forming complexes;
c) separating the sample from the complexes that have arisen;
d) incubating the complexes with plasmin, whereby the non-pathological form of the prion protein is cleaved;
e) separating the cleavage fragments obtained through the incubation with plasmin from the complexes; and
f) detecting un-cleaved prion protein contained in the complexes with detection antibodies.

2. The method according to claim 1, wherein the detection takes place quantitatively in step f).

3. The method according to claim 1, wherein the detection antibodies recognise both the pathological and the non-pathological form of the prion protein.

4. The method according to claim 1, wherein the capture antibody is directed to an epitope of the prion protein which is located in the region of amino acid residues 1-110, if the detection antibody is directed to an epitope which lies outside this region, or the detection antibody is directed to an epitope which is located in the region of amino acid residues 1-110, if the capture antibody is directed to an epitope which lies outside this region.

5. The method according to claim 1, wherein free binding sites of the solid phase are saturated by blocking buffer after step a).

6. The method according to claim 1, wherein the solid phase comprises a microtitre plate or a paramagnetic or non-magnetic bead.

7. The method according to claim 5, wherein the incubation time in step b) amounts to not more than approximately 2 hours.

8. The method according to claim 5, wherein the incubation time in step d) amounts to not more than approximately 30 minutes.

9. The method according to claim 1, further comprising adding aprotinin to the complexes after the incubation with plasmin in step d).

10. The method according to claim 1, wherein washing steps are carried out between the individual steps.

11. The method according to claim 1, wherein the detection antibodies contain biotin, fluorescence markers and/or nanobeads.

12. The method according to claim 1, wherein the detection in step f) comprises an ELISA test.

13. A diagnostic kit for the detection of pathological prions in vitro in a sample, containing:
a) capture antibodies, which are directed both to the pathological (PrP^{Sc}) and the non-pathological form (PrP^{C}) of the prion protein,
b) plasmin; and
c) detection antibodies.

14. The diagnostic kit according to claim 13, wherein the capture antibodies are fixed on a solid phase.

15. The diagnostic kit according to claim 14, wherein the solid phase comprises a microtitre plate or a bead.

16. The diagnostic kit according to claim 13, wherein the detection antibodies recognise both the pathological and the non-pathological form of the prion protein.

17. The diagnostic kit according to claim 13, additionally containing blocking buffer for the saturation of free binding sites of the solid phase, washing buffer and/or aprotinin.

18. The diagnostic kit according to claim 13, wherein the plasmin is dissolved in a buffer solution or lyophilised as a solid.

19. The diagnostic kit according to claim 13, wherein the capture antibody is directed to an epitope of the prion protein which is located in the region of amino acid residues 1-110, if the detection antibody is directed to an epitope which lies outside this region, or the detection antibody is directed to an epitope which is located in the region of amino acid residues 1-110, if the capture antibody is directed to an epitope which lies outside this region.

20. The method of claim 11, wherein the detection antibodies contain nanobeads marked with Europium.
